(19) United States
(12) Patent Application Publication
(10) Pub. No.: US 2024/0415657 A1
(43) Pub. Date: Dec. 19, 2024

(54) FLUID SYSTEMS IN A BODILY IMPLANT

(71) Applicant: Boston Scientific Scimed, Inc., Maple Grove, MN (US)

(72) Inventors: Daniel Walter Myhra, Plymouth, MN (US); Daragh Nolan, Via Youghal (IE); Thomas Sinnott, Wexford (IE); Sai Sardhak Chintaman, Limerick (IE)

(21) Appl. No.: 18/733,271

(22) Filed: Jun. 4, 2024

Related U.S. Application Data
(60) Provisional application No. 63/507,868, filed on Jun. 13, 2023.

(51) Int. Cl. A61F 2/26; A61F 2/00

(52) U.S. Cl. CPC A61F 2/26; A61F 2/004; A61F 2220/0025

(57) ABSTRACT

According to an aspect, an implant, includes an inflatable member; a pump assembly, the pump assembly being configured to facilitate a transfer of fluid to and from the inflatable member; a tubular member extending from the pump assembly; a base member coupled to the pump assembly; and a coupling member configured to be disposed such that a portion of the tubular member is disposed between the base member and the coupling member to facilitate a coupling of the tubular member to the pump assembly.

### CROSS-REFERENCE TO RELATED APPLICATION

This application claims priority to U.S. Provisional Patent Application No. 63/507,868, filed on Jun. 13, 2023, entitled “FLUID SYSTEMS IN A BODILY IMPLANT”, the disclosure of which is incorporated by reference herein in its entirety.

### TECHNICAL FIELD

This disclosure relates generally to bodily implants and more specifically to bodily implants that include fluid systems and inflatable members.

### BACKGROUND

Bodily implants with fluid systems and inflatable members may be placed within a body of a patient for a variety of reasons. For example, one treatment for male erectile dysfunction is the implantation of a penile prosthesis that mechanically erects the penis. Additionally, some patients phalloplasty patients may receive a penile prosthesis. In another example, one treatment for urinary incontinence is the implantation of an artificial urinary sphincter. Such penile prosthesis and artificial urinary sphincters may include an inflatable member or members and a fluid system.

### SUMMARY

According to an aspect, an implant, includes an inflatable member; a pump assembly, the pump assembly being configured to facilitate a transfer of fluid to and from the inflatable member; a tubular member extending from the pump assembly; a base member coupled to the pump assembly; and a coupling member configured to be disposed such that a portion of the tubular member is disposed between the base member and the coupling member to facilitate a coupling of the tubular member to the pump assembly.

In some implementations, the base member includes a projection portion configured to be disposed within a lumen defined by the tubular member. In some implementations, the coupling member is configured to be disposed such that a portion of the coupling member is disposed between the base member and the tubular member.

In some implementations, the tubular member extends between the pump assembly and the inflatable member. In some implementations, the implant includes a reservoir configured to store fluid. In some implementations, the implant includes a reservoir configured to store fluid, the tubular member extending between the pump assembly and the reservoir.

In some implementations, the base member includes an engagement member, the coupling member includes an engagement member configured to engage the engagement member of the base member to couple the coupling member to the base member. In some implementations, the base member defines a cavity, a portion of the coupling member being disposed within the cavity defined by the base member. In some implementations, the base member defines a cavity, a portion of the coupling member being disposed within the cavity defined by the base member, a portion of the tubular member being disposed within the cavity defined by the base member. In some implementations, the base member defines a cavity and includes an engagement member extending into the cavity, the coupling member includes an engagement member configured to engage the engagement member of the base member to couple the coupling member to the base member. In some implementations, the base member includes an engagement member, the coupling member includes a first engagement member and a second engagement member, the first engagement member of the coupling member is configured to engage a first portion of the engagement member of the base member, the second engagement member of the coupling member is configured to engage a second portion of the engagement member of the base member.

In some implementations, the coupling member includes a support portion configured to contact a portion of the tubular member when the tubular member is placed in a non-linear configuration. In some implementations, the coupling member includes a funnel shaped support portion. In some implementations, the coupling member includes a funnel shaped support portion configured to contact a portion of the tubular member when the tubular member is placed in a non-linear configuration. In some implementations, the pump assembly includes a housing having a funnel shaped support portion configured to contact a portion of the tubular member when the tubular member is placed in a non-linear configuration.

In some implementations, the implant includes a support member coupled to a portion of the tubular member.

According to another aspect, a bodily implant includes a housing; a tubular member extending from the housing; a base member coupled to the housing and defining a cavity; and a coupling member, a portion of the coupling member being configured to be disposed within the cavity defined by the base member.

In some implementations, a portion of the tubular member is configured to be disposed within the cavity defined by the base member.

In some implementations, the base member includes a projection, at least a portion of the projection being configured to be disposed within a lumen defined by the tubular member. In some implementations, the base member includes an engagement member, the coupling member includes an engagement member configured to engage the engagement member of the base member to couple the coupling member to the base member.

### BRIEF DESCRIPTION OF THE DRAWINGS

FIG. 1 schematically illustrates an implant according to an implementation.

FIG. 2 illustrates an implant according to an embodiment.

FIG. 3 illustrates an implant according to an embodiment.

FIG. 4 is a perspective view of a pump assembly according to an implementation.

FIGS. 5 and 6 are side views of the pump assembly and a base member.

FIGS. 7 and 8 are perspective views of the pump assembly, the base member, and a tubular member.

FIGS. 9 and 10 are perspective views of the pump assembly, the base member, the tubular member, and a coupling member.

FIGS. 11 and 12 are cross-sectional views of the pump assembly, the base member, the tubular member, and the coupling member.

FIG. 13 is an exploded view of the pump assembly, the base member, the tubular member, and the coupling member.

FIGS. 14 and 15 illustrate the base member, the tubular member, and the coupling member.

FIGS. 16-21 illustrate a pump assembly, a base member, a tubular member, and a coupling member according to another implementation.

FIGS. 22-24 illustrate a pump assembly, a base member, a tubular member, and a coupling member according to another implementation.

FIGS. 25-28 illustrate a pump assembly, a base member, a tubular member, and a coupling member according to another implementation.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as penile prostheses, artificial sphincters, or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of a bodily implant. For example, the bodily implant may have improved reliability or improved implantation procedures.

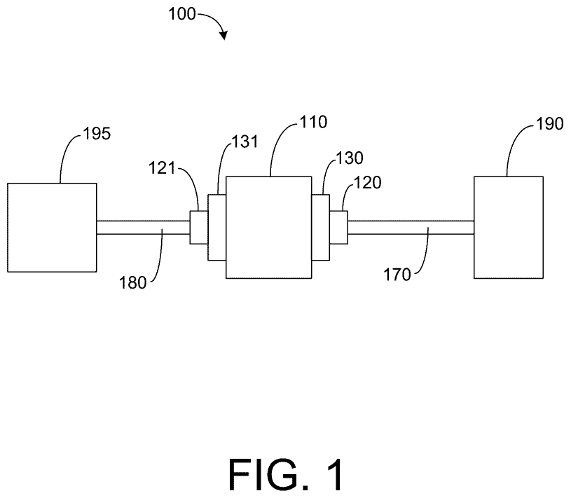

FIG. 1 schematically illustrates a bodily implant 100 according to an embodiment. In some embodiments, the bodily implant 100 may be a penile implant. In other embodiments, the bodily implant 100 may be an artificial urinary sphincter. In yet other embodiments, the bodily implant 100 is a different type of bodily implant.

The illustrated embodiment includes a pump assembly 110, an inflatable member 190, and a reservoir 195. The pump assembly 110 is operatively and fluidically coupled to the reservoir 195 via tubular member 170. The pump assembly 110 is operatively and fluidically coupled to the reservoir 195 via the tubular member 180.

The reservoir 195 is configured to hold or store a fluid. The pump assembly 110 is configured to move fluid from the reservoir 195 to the inflatable member 190 and from the inflatable member 190 to the reservoir 195. The pump assembly 110 may include electronic pump components or manual pump components. The inflatable member 190 is configured to be placed in an inflated state or configuration and in a deflated state or configuration. In some embodiments, the inflatable member 190 is configured to place pressure on a portion of the body when the inflatable member 190 is in the inflated state or configuration.

In the illustrated embodiment, the tubular member 170 is coupled to and extends between the pump assembly 110 and the inflatable member 190. The tubular member 170 is configured to convey fluid between the pump assembly 110 and the inflatable member 190.

The bodily implant 100 includes a base member 130 and a coupling member 120. The base member 130 is configured to be coupled to the pump assembly 110, such as to a housing or a surface of the pump assembly 110. In some implementations, the coupling member 120 is configured to engage the base member 130 to help couple the tubular member 170 to the pump assembly 110.

In the illustrated embodiment, the tubular member 180 is coupled to and extends between the pump assembly 110 and to the reservoir 195. The tubular member 180 is configured to convey fluid between the pump assembly 110 and the reservoir 195.

The bodily implant 100 includes a base member 131 and a coupling member 121. The base member 131 is configured to be coupled to the pump assembly 110, such as to a housing or a surface of the pump assembly 110. In some implementations, the coupling member 121 is configured to engage the base member 131 to help couple the tubular member 180 to the pump assembly 110.

In use, the bodily implant 100 may be placed within the body of a patient. In some embodiments, the inflatable member 190 may be placed such that it is able to place pressure on a portion of the body of the patient when the inflatable member 190 is in the inflated or expanded configuration.

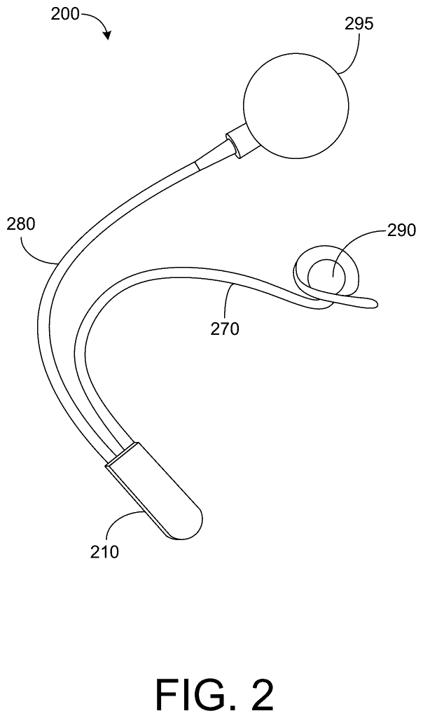

FIG. 2 illustrates a bodily implant 200 according to an embodiment. The implant 200 is an artificial urinary sphincter. The implant 200 includes a pump assembly 210, an inflatable member 290, and a reservoir 295. The pump assembly 210 is operatively and fluidically coupled to the inflatable member 290 via tubular member 270. The pump assembly 210 is operatively and fluidically coupled to the reservoir 295 via the tubular member 280.

The reservoir 295 is configured to hold or store a fluid. The pump assembly 210 is configured to move fluid from the reservoir 295 to the inflatable member 290 and from the inflatable member 290 to the reservoir 295. The pump assembly 210 may include electronic pump components or manual pump components. The inflatable member 290 is configured to be placed in an inflated state or configuration and in a deflated state or configuration. In some embodiments, the inflatable member 290 is configured to place pressure on a portion of the body when the inflatable member 290 is in the inflated state or configuration.

In the illustrated embodiment, the tubular member 270 is coupled to and extends between the pump assembly 210 and the inflatable member 290. The tubular member 270 is configured to convey fluid between the pump assembly 210 and the inflatable member 290. In some implementations, bodily implant 200 includes a base member and a coupling member. The base member is configured to be coupled to the pump assembly 210, such as to a housing or a surface of the pump assembly 210. In some implementations, the coupling member is configured to engage the base member to help couple the tubular member 270 to the pump assembly 210.

In the illustrated embodiment, the tubular member 280 is coupled to and extends between the pump assembly 210 and the reservoir 295. The tubular member 280 is configured to convey fluid between the pump assembly 210 and the reservoir 295. In some implementations, bodily implant 200 includes a second base member and a second coupling member. The second base member is configured to be coupled to the pump assembly 210, such as to a housing or a surface of the pump assembly 210. In some implementations, the second coupling member is configured to engage the second base member to help couple the tubular member 280 to the pump assembly 210.

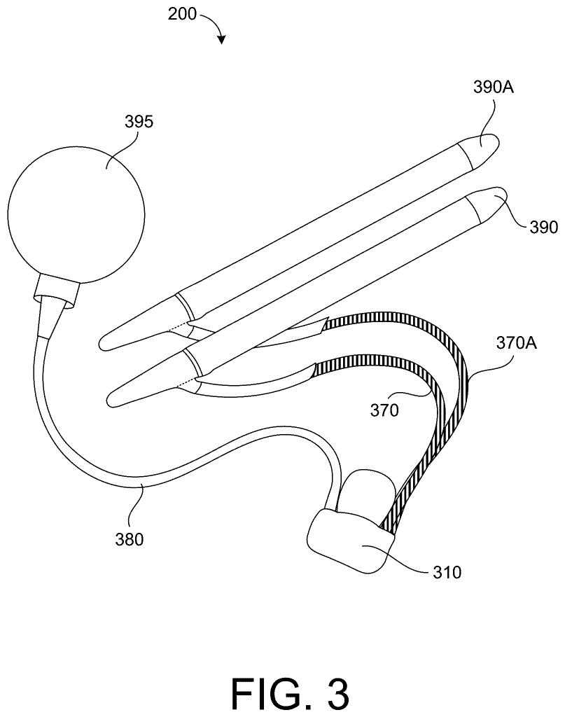

FIG. 3 illustrates a bodily implant 300 according to an embodiment. The implant 300 is penile implant. The implant 300 includes a pump assembly 310, an inflatable member 390, and a reservoir 395. The implant also includes a second inflatable member 390A. The pump assembly 310 is operatively and fluidically coupled to the inflatable member 390A via tubular member 370A. The pump assembly 310 is operatively and fluidically coupled to the inflatable member 390 via tubular member 370. In some implementations, a tubular member with a single end coupled to the pump assembly may couple the pump assembly to both inflatable members (such as a tubular member that includes a second branch or has a Y shape). The pump assembly 310 is operatively and fluidically coupled to the reservoir 395 via the tubular member 380.

The reservoir 395 is configured to hold or store a fluid. The pump assembly 310 is configured to move fluid from the reservoir 395 to the inflatable members 390 and 390A and from the inflatable members 390 and 390A to the reservoir 395. The pump assembly 310 may include electronic pump components or manual pump components. The inflatable members 390 and 390A are configured to be placed in inflated states or configurations and in deflated states or configurations. In some embodiments, the inflatable members 390 and 390A are configured to place pressure on a portion of the body when the inflatable members 390 and 390A are in the inflated states or configurations.

In the illustrated embodiment, the tubular member 370 is coupled to the pump assembly 310 and to the inflatable member 390. The tubular member 370 is configured to convey fluid between the pump assembly 310 and the inflatable member 390. In some implementations, bodily implant 300 includes a base member and a coupling member. The base member is configured to be coupled to the pump assembly 310, such as to a housing or a surface of the pump assembly 310. In some implementations, the coupling member is configured to engage the base member to help couple the tubular member 370 to the pump assembly 310.

In the illustrated embodiment, the tubular member 370A is coupled to the pump assembly 310 and to the inflatable member 390A. The tubular member 370A is configured to convey fluid between the pump assembly 310 and the inflatable member 390A. In some implementations, bodily implant 300 includes a base member and a coupling member. The base member is configured to be coupled to the pump assembly 310, such as to a housing or a surface of the pump assembly 310. In some implementations, the coupling member is configured to engage the base member to help couple the tubular member 370A to the pump assembly 310.

In the illustrated embodiment, the tubular member 380 is coupled to the pump assembly 310 and to the reservoir 395. The tubular member 380 is configured to convey fluid between the pump assembly 310 and the reservoir 395. In some implementations, bodily implant 300 includes a base member and a coupling member. The base member is configured to be coupled to the pump assembly 310, such as to a housing or a surface of the pump assembly 310. In some implementations, the coupling member is configured to engage the base member to help couple the tubular member 380 to the pump assembly 310.

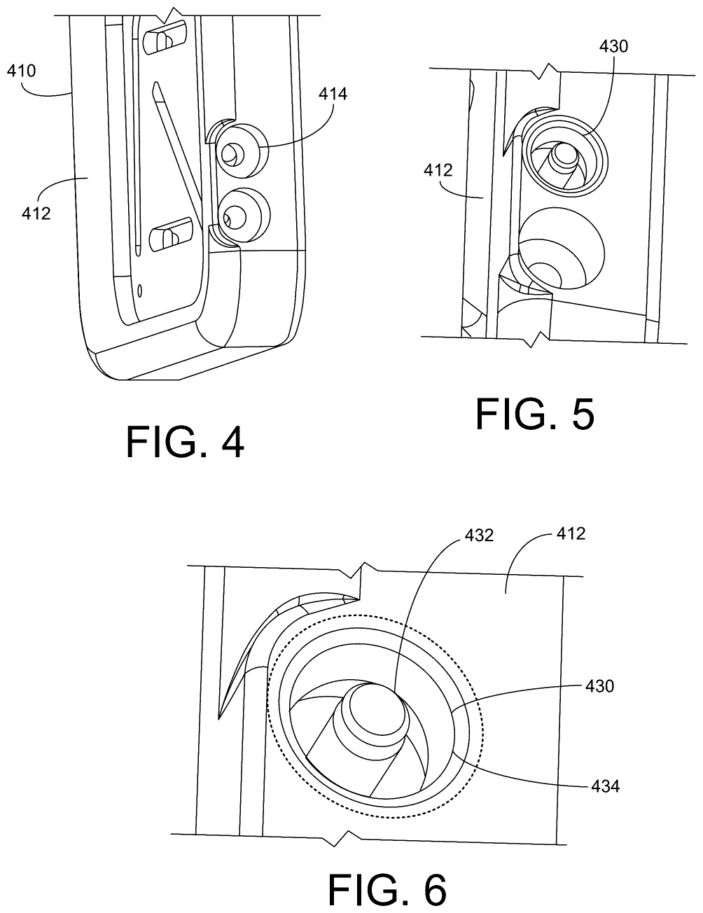

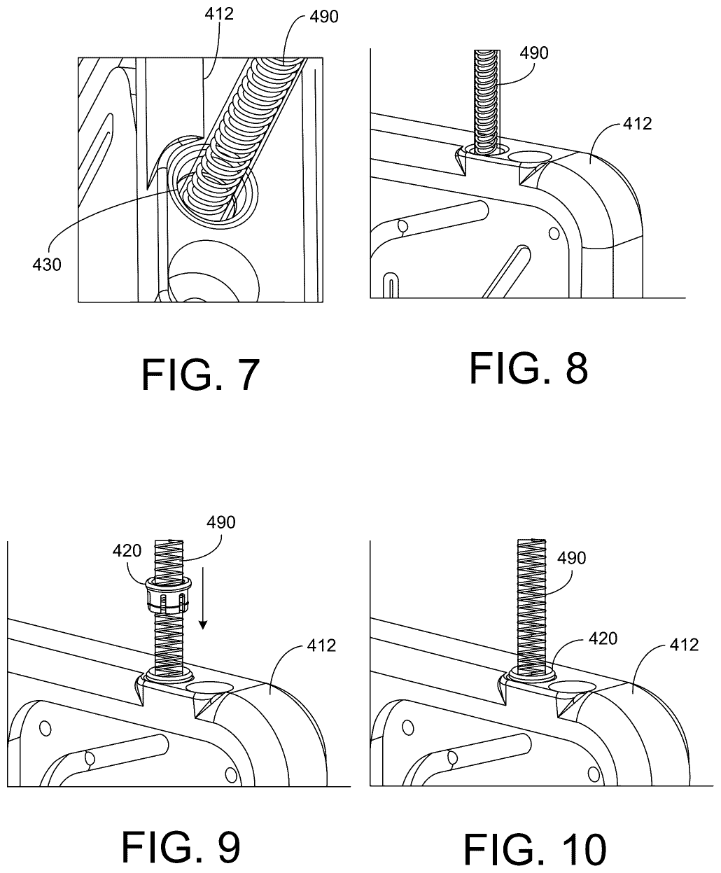

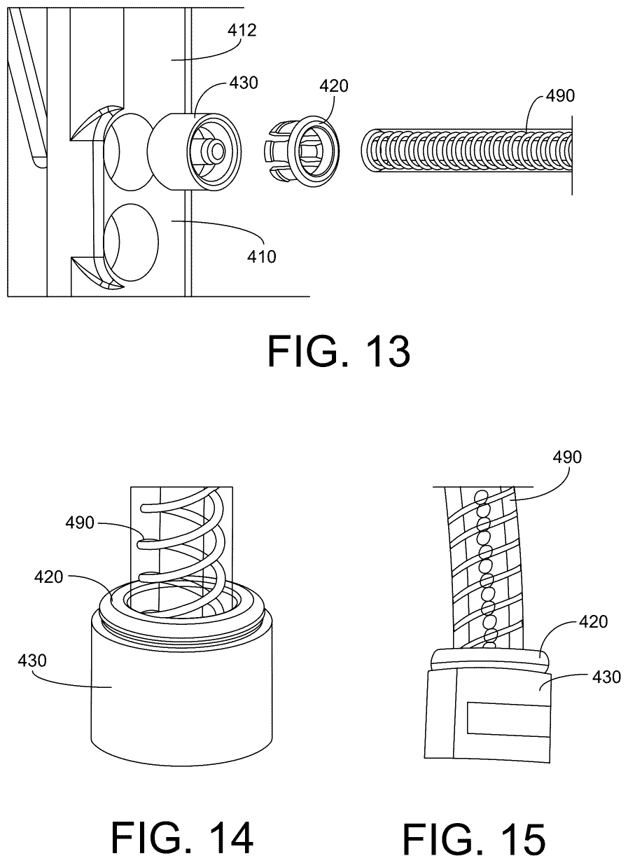

FIGS. 4-15 illustrate a pump assembly 410, a base member 430, a tubular member 490, and a coupling member 420 according to an implementation. FIG. 13 is an exploded view of the implementation. As illustrated the tubular member 490 may be used to fluidically couple the pump assembly 410 to an inflatable member or to a fluid reservoir. In some implementations, for example, as described above, the bodily implant may include several tubular members that fluidically couple the pump assembly to other components, such as a fluid reservoir or an inflatable member. While only one tubular member 490 and its coupling to the pump assembly 410 is illustrated and described in detail, it should be understood that such a coupling may be used to couple other or more than one tubular member to the pump assembly or to other portions or components of the device.

The pump assembly includes a housing 412. In the illustrated implementation, the base member 430 is coupled to the housing 412 of the pump assembly 410 within a recess 414 defined by the housing 412. In some implementations, the base member 430 may be coupled to a surface of the housing, such as a non-recessed outer surface of the housing. In some implementations, the housing 412 and the base member 430 are formed of metal and the base member 430 is coupled to the housing 412 via a welding, such as a laser welding, process. In other implementations, the base member or the housing or both are made of different materials. In other implementations, the base member is coupled to the housing via a different coupling mechanism, such as an adhesive. In yet another implementation, the housing and the base member may be unitarily or monolithically formed. In such an implementation, the housing may be formed or machined to have the structure and form of the housing and the base member when the base member is coupled to the housing.

In the illustrated implementation, the recess is in communication with a lumen or opening 416 defined in the housing 412. The lumen or opening 416 is in communication with pumps and valves within the pump assembly to facilitate fluid flow to and from the pump assembly via that lumen or opening 416.

The base member 430 includes a projection 432. The projection 432 defines a lumen 433. When coupled to the housing 412, the lumen 433 defined by the base member 430 is in fluid communication with the lumen or opening 416 defined by the housing 412.

The tubular member 490 may be placed over the projection 432 of the base member 430. In the illustrated implementation, the projection 432 of the base member 430, or a portion of the projection 432 of the base member 430, extends into a lumen 492 defined by the tubular member 490. In some implementations, the tubular member 490 is a kink resistant tubing. In other implementations, the tubular member is a different type of member or is formed of different materials.

As best illustrated in FIGS. 9 and 10, the coupling member 420 is moved or slid along an outer surface of the tubular member 490. In the illustrated implementation, the coupling member 420 defines a lumen 422. The tubular member 490 is disposed within the lumen 422. The coupling member 420 may be moved towards the pump assembly 410 until the coupling member 420 engages the base member 430.

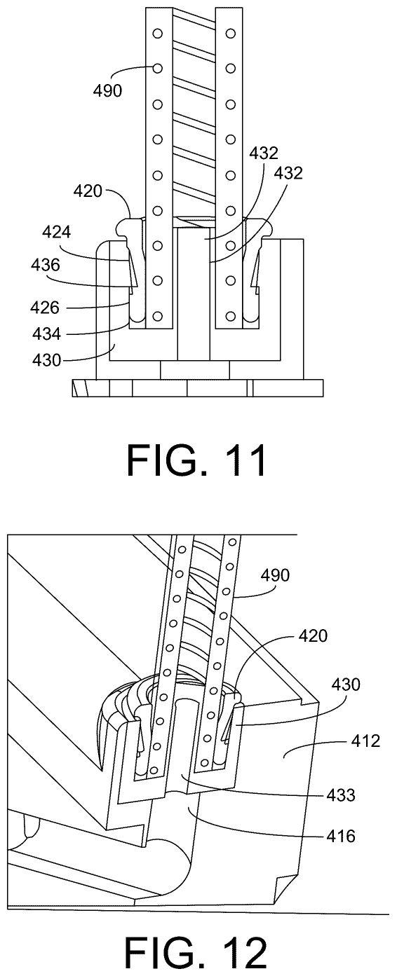

As best illustrated in FIGS. 11 and 12, base member 430, the coupling member 420, and the tubular member 490 are disposed such that a portion of the coupling member 420 is disposed between a portion of the tubular member 490 and a portion of the base member 430. Additionally, a portion of the tubular member 490 is disposed between a portion of the projection 432 of the base member 430 and a portion of the coupling member 420. In the illustrated implementation, the tubular member 490 is thereby coupled to the pump assembly 410 or the housing 412 of the pump assembly 410.

In the illustrated implementation, the coupling member 420 is configured to engage the base member 430 and couple to the base member 430. Specifically, an inner surface 434 of the base member 430 includes a projection or coupling portion 436. The projection or coupling portion 436 of the base member 430 is configured to engage a projection or coupling portion 426 of an outer surface 424 of the coupling member 420 to couple the coupling member 420 to the base member 430. The coupling member may include more than one projection or coupling portion. Similarly, the base member may include more than one projection or coupling portion. In other implementations, the coupling member attaches or couples to the base member via a different mechanism. In some implementations, the coupling member 420 and the base member 430 are formed of biocompatible materials, such as a polymer material. In some implementations, the materials that form the coupling member 420 and the base member 430 allow the members to be fixedly coupled to each other.

FIG. 13 is an exploded view of the components. FIGS. 14 and 15 illustrate the coupler 420 coupled to the base member 430 and to the tubular member 490.

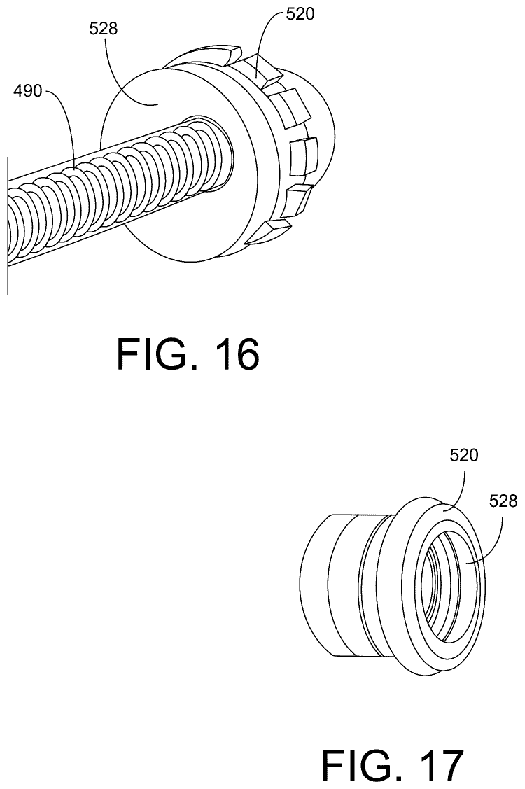

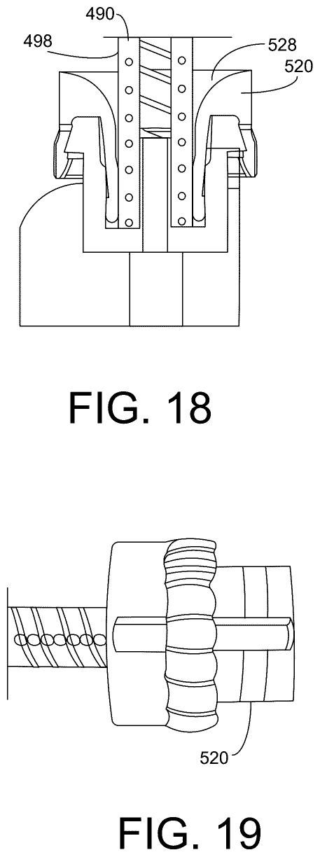

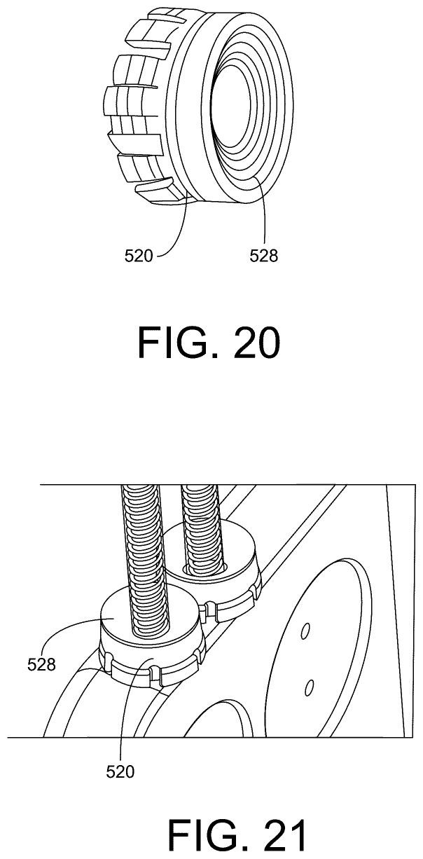

FIGS. 16-21 illustrate a coupling member 520 according to an implementation. The coupling member includes a curved or flared surface 528. The curved or flared surface 528 is configured to contact the tubular member 490, such as an outer surface 498 of the tubular member. For example, in some implementations, the curved or flared surface 528 is configured to contact the outer surface 498 of the tubular member 490 when the tubular member 490 is curved or bent into a non-linear configuration to provide support to the tubular member 490. In some implementations, the curved or flared surface 528 may be configured to help prevent the tubular member 490 from becoming kinked or broken.

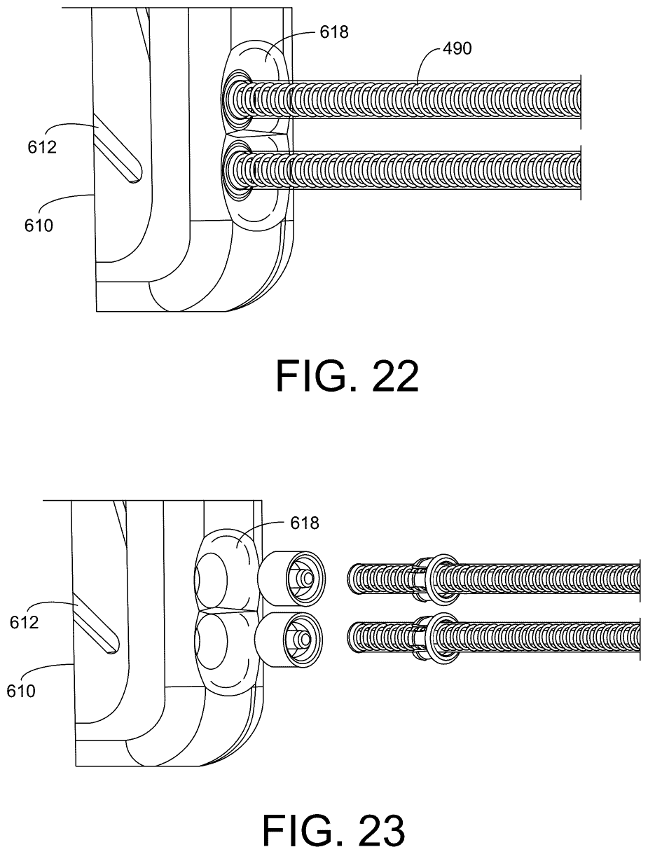

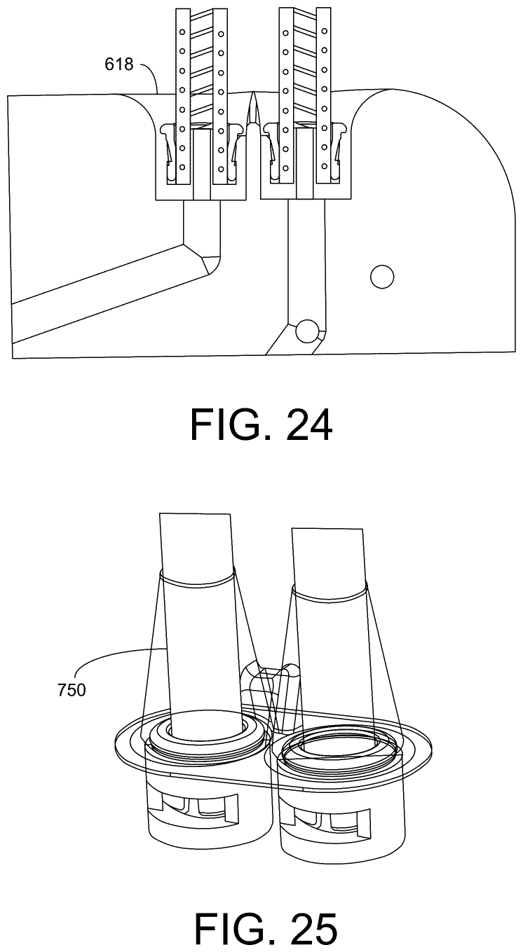

FIGS. 22-24 illustrate a housing 612 of a pump assembly 610 according to an implementation. The housing 612 includes a curved or flared surface 618. The curved or flared surface 618 is configured to contact the tubular member 490, such as an outer surface 498 of the tubular member. For example, in some implementations, the curved or flared surface 618 is configured to contact the outer surface 498 of the tubular member 490 when the tubular member 490 is curved or bent into a non-linear configuration to provide support to the tubular member 490. In some implementations, the curved or flared surface 618 may be configured to help prevent the tubular member 490 from becoming kinked or broken.

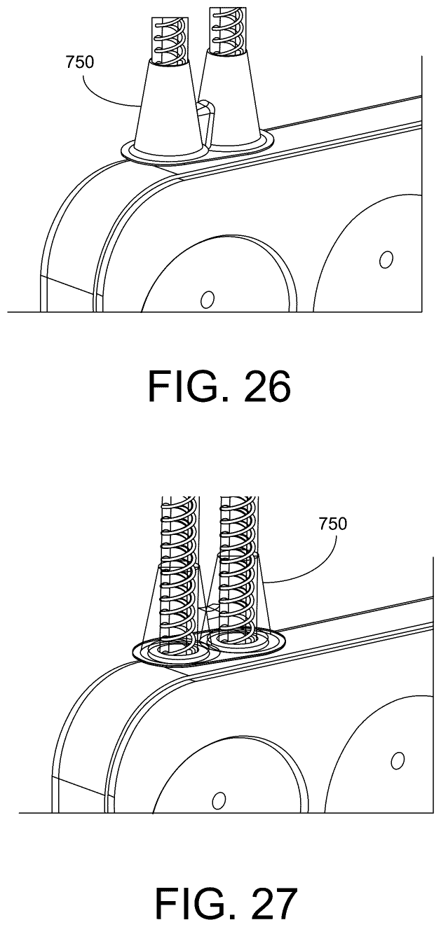

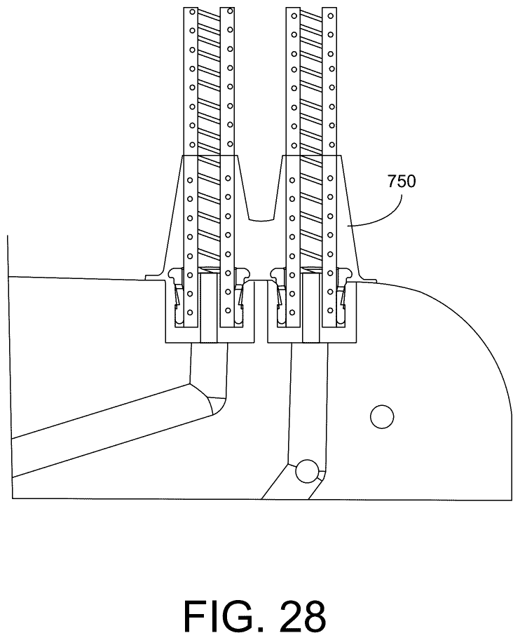

FIGS. 25-28 illustrate a system according to another implementation. The system includes a support member 750. The support member 750 is configured to contact the tubular member to provide support to the tubular member. In some implementations, the support member 750 may be configured to help prevent the tubular member from becoming kinked or broken.

In the illustrated implementation, the support member 750 fits over or is coupled to two tubular members and is disposed on top of the pump assembly housing. In implementations, the support member may be located at a different location along the tubular member.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implant, comprising:
an inflatable member;
a pump assembly, the pump assembly being configured to facilitate a transfer of fluid to and from the inflatable member;
a tubular member extending from the pump assembly;
a base member coupled to the pump assembly; and
a coupling member configured to be disposed such that a portion of the tubular member is disposed between the base member and the coupling member to facilitate a coupling of the tubular member to the pump assembly.

2. The implant of claim 1, wherein the base member includes a projection portion configured to be disposed within a lumen defined by the tubular member.

3. The implant of claim 1, wherein the coupling member is configured to be disposed such that a portion of the coupling member is disposed between the base member and the tubular member.

4. The implant of claim 1, wherein the tubular member extends between the pump assembly and the inflatable member.

5. The implant of claim 1, further comprising:
a reservoir configured to store fluid.

6. The implant of claim 1, further comprising:
a reservoir configured to store fluid, the tubular member extending between the pump assembly and the reservoir.

7. The implant of claim 1, wherein the base member includes an engagement member, the coupling member includes an engagement member configured to engage the engagement member of the base member to couple the coupling member to the base member.

8. The implant of claim 1, wherein the base member defines a cavity, a portion of the coupling member being disposed within the cavity defined by the base member.

9. The implant of claim 1, wherein the base member defines a cavity, a portion of the coupling member being disposed within the cavity defined by the base member, a portion of the tubular member being disposed within the cavity defined by the base member.

10. The implant of claim 1, wherein the base member defines a cavity and includes an engagement member extending into the cavity, the coupling member includes an engagement member configured to engage the engagement member of the base member to couple the coupling member to the base member.

11. The implant of claim 1, wherein the base member includes an engagement member, the coupling member includes a first engagement member and a second engagement member, the first engagement member of the coupling member is configured to engage a first portion of the engagement member of the base member, the second engagement member of the coupling member is configured to engage a second portion of the engagement member of the base member.

12. The implant of claim 1, wherein the coupling member includes a support portion configured to contact a portion of the tubular member when the tubular member is placed in a non-linear configuration.

13. The implant of claim 1, wherein the coupling member includes a funnel shaped support portion.

14. The implant of claim 1, wherein the coupling member includes a funnel shaped support portion configured to contact a portion of the tubular member when the tubular member is placed in a non-linear configuration.

15. Th implant of claim 1, wherein the pump assembly includes a housing having a funnel shaped support portion configured to contact a portion of the tubular member when the tubular member is placed in a non-linear configuration.

16. The implant of claim 1, further comprising a support member coupled to a portion of the tubular member.

17. A bodily implant, comprising:
a housing;
a tubular member extending from the housing;
a base member coupled to the housing and defining a cavity; and
a coupling member, a portion of the coupling member being configured to be disposed within the cavity defined by the base member.

18. The bodily implant of claim 17, wherein a portion of the tubular member is configured to be disposed within the cavity defined by the base member.

19. The bodily implant of claim 17, wherein the base member includes a projection, at least a portion of the projection being configured to be disposed within a lumen defined by the tubular member.

20. The implant of claim 17, wherein the base member includes an engagement member, the coupling member includes an engagement member configured to engage the engagement member of the base member to couple the coupling member to the base member.
